(19) United States
(12) Plant Patent Application Publication
(10) Pub. No.: US 2021/0400858 P1
(43) Pub. Date: Dec. 23, 2021

(54) Banana plant named 'QCAV-4'

(50) ;

(71) Applicant: Queensland University of Technology, Brisbane (AU)

(72) Inventors: James Dale, Brisbane (AU); Robert Harding, Brisbane (AU); Harjeet Khanna, Brisbane (AU); Anthony James, Brisbane (AU); Jennifer Kleidon, Brisbane (AU); Mark Smith, Darwin (AU); Upendra Shekhawat, Brisbane (AU)

(73) Assignee: Queensland University of Technology, Brisbane (AU)

(21) Appl. No.: 16/873,740

(22) Filed: Jun. 19, 2020

(51) Int. Cl. A01H 6/00

(52) U.S. Cl. PLT/160

(57) ABSTRACT

A new banana cultivar ‘QCAV-4’ is provided that, when under significant disease pressure, remains largely free from infection by Fusarium wilt tropical race 4 (TR4). In the absence of such significant disease pressure, ‘QCAV-4’ appears to be essentially phenotypically identical to the wild type parent Cavendish Grand Nain. This includes in relation to immature and mature plant characteristics, fruit characteristics, and yield.

Latin name of the genus and species of the plant claimed: Musa acuminata.

Variety denomination: ‘QCAV-4’.

### BACKGROUND OF THE INVENTION

The present invention relates to a new and distinct cultivar of banana plant named ‘QCAV-4’. The new plant resulted from transformation of parent Cavendish Grand Nain (unpatented) by T-DNA insertion and selection. A resulting transgenic plant named ‘QCAV-4’ was selected when growing in a cultivated area in Lambells Lagoon, Northern Territory, Australia.

### BRIEF SUMMARY OF THE INVENTION

‘QCAV-4’ is a transgenic cultivar produced from Cavendish Grand Nain. For initial transformation, embryogenic cell suspensions (ECS) were generated from immature male flowers from the bell (flower) of Cavendish Grand Nain. The bells were collected in North Queensland, Australia and indexed for virus infection. The ECS were transformed using Agrobacterium mediated transformation. The transformation cassette included a selectable marker gene, neomycin phosphotransferase (NPT II). The resistance gene was a gene isolated from Musa acuminata subsp. malaccensis which is resistant to Fusarium wilt tropical race 4 (TR4). The resistance gene was under the control of the nos promoter. Potentially transformed cells were placed on kanamycin to select NPT II resistant cells. These were then regenerated into whole plantlets and multiplied. Presence of the transgenes were confirmed by PCR. Multiplied plantlets were transferred to a farm in Lambells Lagoon, Northern Territory, Australia and acclimatized in a screenhouse. These plants together with appropriate controls were planted into a plot where Cavendish bananas had been previously grown and had been severely affected by Fusarium wilt TR4. The plot was “seeded” further with pseudostem segments from infected Cavendish plants. Plants were regularly inspected for TR4 symptoms over a three-year period. Multiple independent transformed lines demonstrated strong resistance to TR4 as compared to the parental Cavendish Grand Nain, which is highly susceptible. Morphological characteristics of plants and fruit were assessed, bunch weight was measured, and molecular analysis was performed. One line was selected based on morphological and molecular analysis, and named ‘QCAV-4’.

The ‘QCAV-4’ cultivar is distinguished from other banana varieties, including the parent, by having a strong resistance phenotype to Fusarium wilt tropical race 4 (TR4). It is substantially phenotypically identical to its parent in the absence of disease pressure.

Asexual reproduction of ‘QCAV-4’ by tissue culture in Brisbane City, Queensland, Australia in combination with field assessment in Lambells Lagoon, Northern Territory, Australia, shows that the foregoing characteristic resistance to Fusarium wilt TR4 reproduces true to type.

The following detailed description concerns progeny lines asexually propagated from the original line by tissue culture.

### BRIEF DESCRIPTION OF THE DRAWINGS

FIG. 1 is a digital image of Southern blot analysis of wild type Cavendish Grand Nain (parent) and ‘QCAV-4’, showing endogenous RGA2 copies and transgenic RGA2 insertions.

FIG. 2 is a schematic diagram of transgenic RGA2 T-DNA insertions on chromosome 6 of ‘QCAV-4’ as determined by genome sequencing and analysis. The model is based on Geneious Blast enabled virtual genome walking outwards from the left and right T-DNA borders and mapping the reads using varying stringency parameters on vector sequence.

FIGS. 3-4 are photographs showing ‘QCAV-4’ at adult stage.

FIG. 5 is photograph showing ‘QCAV-4’ at adult stage and developing fruit.

FIGS. 6-7 are photographs showing ‘QCAV-4’ at adult stage.

The colors of an illustration of this type may vary with lighting and other conditions under which conditions and, therefore, color characteristics of this new cultivar should be determined with reference to the observations described herein, rather than from these illustrations alone.

### DETAILED DESCRIPTION

The following detailed description of ‘QCAV-4’ is based on observations of plants that are approximately 25 months old. The ‘QCAV-4’ plants have been observed growing in a cultivated area in Lambells Lagoon, Northern Territory, Australia. Certain characteristics of this cultivar, such as growth and color, may change with changing environmental conditions (such as, light, temperature, moisture, nutrient availability, or other factors). Color descriptions and other terminology are used in accordance with their ordinary dictionary descriptions, unless the context clearly indicates otherwise.
Botanical description:
   Scientific name.—Musa acuminata ‘QCAV-4’.
   Parentage.—Cavendish Grand Nain.
   Plant.—In the absence of significant disease pressure, ‘QCAV-4’ appears to be essentially phenotypically identical to the wild type parent ‘Cavendish Grand Nain’ (unpatented) (Table 1). This includes in relation to immature and mature plant characteristics, fruit characteristics, and yield.

TABLE 1
Comparison of ‘QVAC-4’ to parent ‘Cavendish Grand Nain’*

| Organ/Plant Part: Context | ‘QVAC-4’ | ‘Cavendish Grand Nain’ |
|---|---|---|
| Ploidy: | triploid | triploid |
| Pseudostem: overlapping of leaf sheaths | weak | weak |
| Pseudostem: tapering | absent or weak | absent or weak |
| Pseudostem: colour | purple | purple |
| Pseudostem: anthocyanin colouration | medium to strong | medium |
| Pseudostem: colour of inner side of basal sheath | purple | purple |
| Plant: compactness of crown | compact | compact |
| Plant: growth habit | drooping | drooping |
| Petiole: attitude of wings at base | curved outwards | curved outwards |
| Leaf blade: colour of midrib on lower side | green | green |
| Leaf blade: shape of base | both sides acute | both sides acute |
| Leaf blade: waxiness on lower side | medium | weak to medium |
| Leaf blade: width | broad | broad |
| Leaf blade: glossiness of upper side | absent | absent |
| Peduncle: diameter | large | large |
| Peduncle: pubescence | present | present |
| Peduncle: curvature | medium to strong | medium to strong |
| Bunch: length | long | long |
| Bunch: shape | cylindrical | cylindrical |
| Bunch: attitude of fruits | moderately | moderately |
| | turned up | turned up |
| Bunch: compactness | medium | medium |
| Bunch: number of hands | many | many |
| Rachis: attitude of male part | vertical | vertical |
| Rachis: prominence of scars | weak | weak |
| Rachis: persistence of bracts | absent or weak | absent or weak |
| Rachis: persistence of hermaphrodite flowers | present | present |
| | greenish yellow | greenish yellow |
| Fruit: colour of peel (before maturity) | (RHS 141C) | (RHS 141C) |
| Fruit: persistence of floral organs | present | present |
| Male inflorescence: persistence | present | present |
| Male inflorescence: shape | narrow ovate | narrow ovate |
| Male inflorescence: opening of bracts | closed or | closed or |
| | slightly open | slightly open |
| Bract: colour of inner side | orange red | orange red |
| Bract: shape of apex | broad acute | broad acute |

*For most characteristics, 4-5 individual plants were assessed.

However, a clear phenotype is observable under pressure from Fusarium wilt tropical race 4 (TR4).

In March 2018, an expanded field trial was planted which included 50 replicates of each of the four events from Trial 1, in 10×5 randomized plot design. In addition to recording disease incidence, detailed agronomic information such as bunch weight, number of fingers on the top hand and crop cycling time is also collected. Since the trial began, agronomic data for the plant crop and at least two ratoon crops were collected. The trial is ongoing. Based on the results of these field trials and molecular characterisation, ‘QCAV-4’ was selected.

The disease status of plants is assessed by the presence of characteristic disease symptoms (both external and internal) and by molecular testing of vascular tissue for the presence of the fungal pathogen TR4. The plants are inspected on a weekly basis and plants showing the characteristic external symptoms of the disease identified. About 1-2 weeks later, the pseudostem of these plants is cut and examined for the presence of the highly characteristic internal vascular discolouration associated with TR4 infection. DNA is extracted from the infected vascular tissue, and a highly sensitive PCR test is used to detect the presence of TR4, and this is confirmed by sequencing. The TR4 fungus from discoloured vascular tissue is obtained and DNA extracted and analysed using PCR to confirm the presence of TR4 (and also by sequencing).

As shown in Table 2, ‘QCAV-4’ can remain largely disease-free under the same conditions of TR4 pressure leading to greater than 80% infection rates in wild type ‘Cavendish Grand Nain’.

TABLE 2
Resistance of Plants to Fusarium wilt tropical race 4 (TR4).

| | Number | Number | Percent |
|---|---|---|---|
| Variety | of Plants | Infected | Infected |
| ‘Cavendish Grand Nain’ | 50 | 32 | 64 |
| ‘Cavendish Williams’ | 50 | 38 | 76 |
| (unpatented) | | | |
| ‘RGA2-5’ (unpatented) | 50 | 14 | 28 |
| ‘RGA2-2’ (unpatented) | 50 | 8 | 16 |
| ‘RGA2-3’ (unpatented) | 50 | 3 | 6 |
| ‘QVAC-4’ | 50 | 0 | 0 |

TABLE 3
Fruit Production Characteristics

| | Plant crop | | | Ratoon 1 | | | Ratoon 2 | | |
|---|---|---|---|---|---|---|---|---|---|
| | | No. of | | | No. of | | | No. of | |
| | Bunch | fingers | | Bunch | fingers | | Bunch | fingers | |
| | weight | (top | Cycle 1 | weight | (top | Cycle 2 | weight | (top | Cycle 3 |
| Line | (kg) | hand) | (days) | ikg ) | hand) | (days) | (kg) | hand) | (days) |
| Grand Nain | 33.1 | 24.0 | 327.3 | 29.9 | 20.0 | 212.4 | 31.7 | 26.5 | 206.6 |
| Williams | 30.7 | 23.0 | 324.7 | 29.8 | 22.2 | 212.9 | 40.4 | 25.9 | 211.8 |
| RGA2-2 | 29.1 | 22.7 | 268.3 | 27.6 | 22.3 | 223.8 | 28.1 | 24.1 | 215.5 |
| RGA2-3 | 30.1 | 24.9 | 328.9 | 23.8 | 20.2 | 200.8 | 29.2 | 26.1 | 211.7 |
| QVAC-4 | 28.1 | 22.5 | 331.1 | 24.3 | 19.7 | 199.2 | 33.7 | 25.3 | 206.9 |
| RGA2-5 | 35.5 | 24.8 | 341.6 | 30.6 | 22.0 | 212.9 | 37.3 | 29.5 | 212.8 |

Height of about 180 to 250 cm—shorter than Giant Cavendish and taller than Dwarf Cavendish cultivars.

Moderate adult pseudostem width.

Relatively large bunch size.

Moderate fruit size.

Solid green leaf colour.
Southern analysis:

Genomic DNA was extracted from ‘QCAV-4’ and wild type (non-transformed) Cavendish Grand Nain. The DNA was digested with a restriction enzyme, electrophoresed through an agarose gel, transferred to a membrane, and probed with a labelled RGA2 probe.

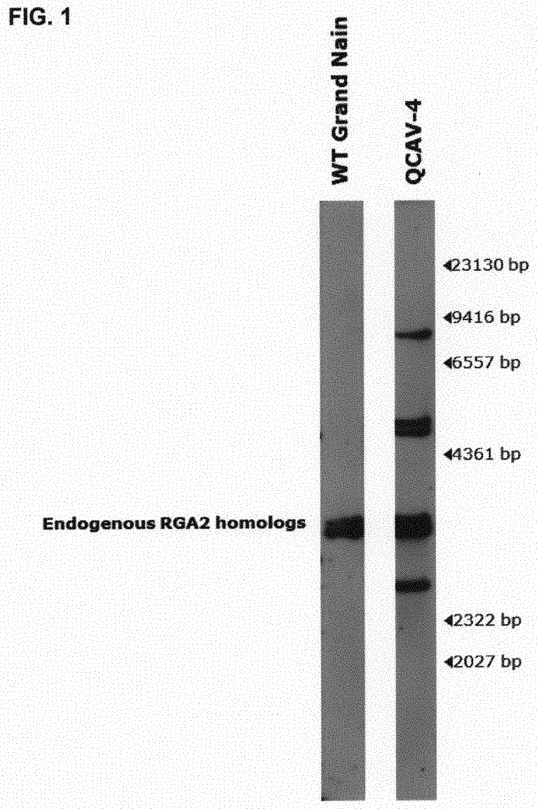

As shown in FIG. 1, Southern analysis was consistent with four transgene copies in ‘QCAV-4’, in addition to the endogenous RGA2 genes. Cavendish Grand Nain is a triploid and it could be expected to have three endogenous copies of RGA2. Two distinct bands were identified in the wild type, indicating that two of three endogenous copies may have migrated together.
Genome sequencing:

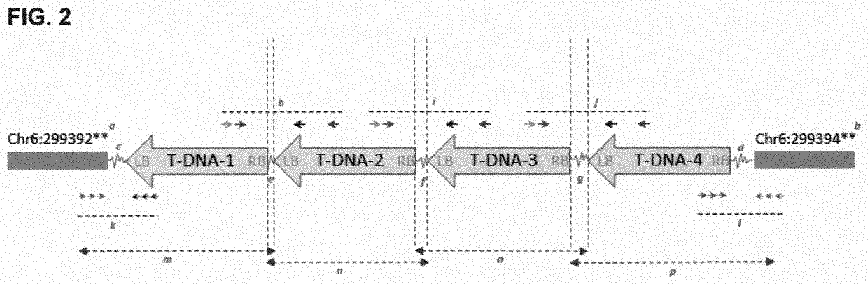

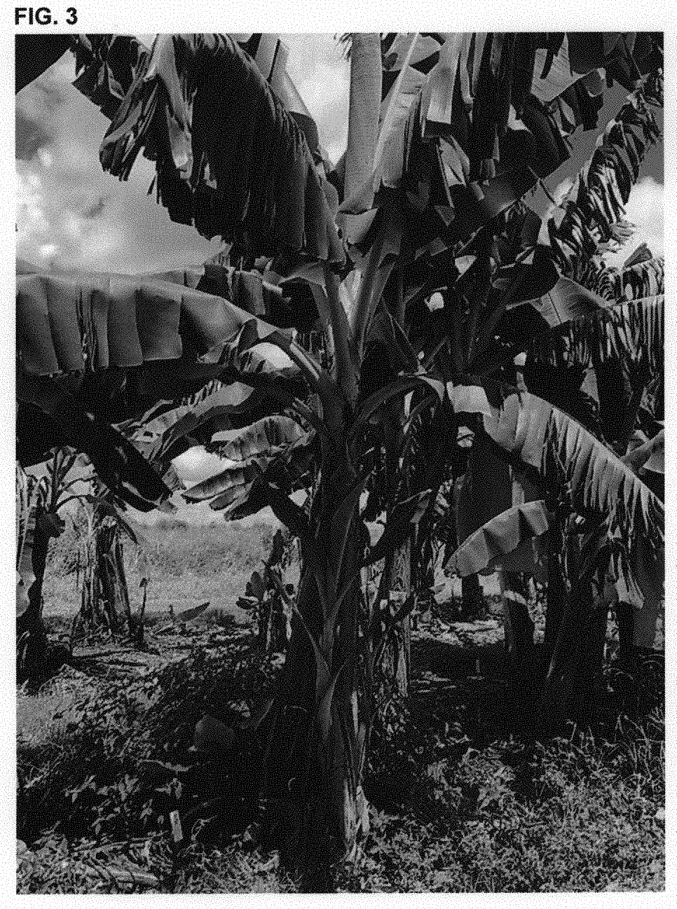

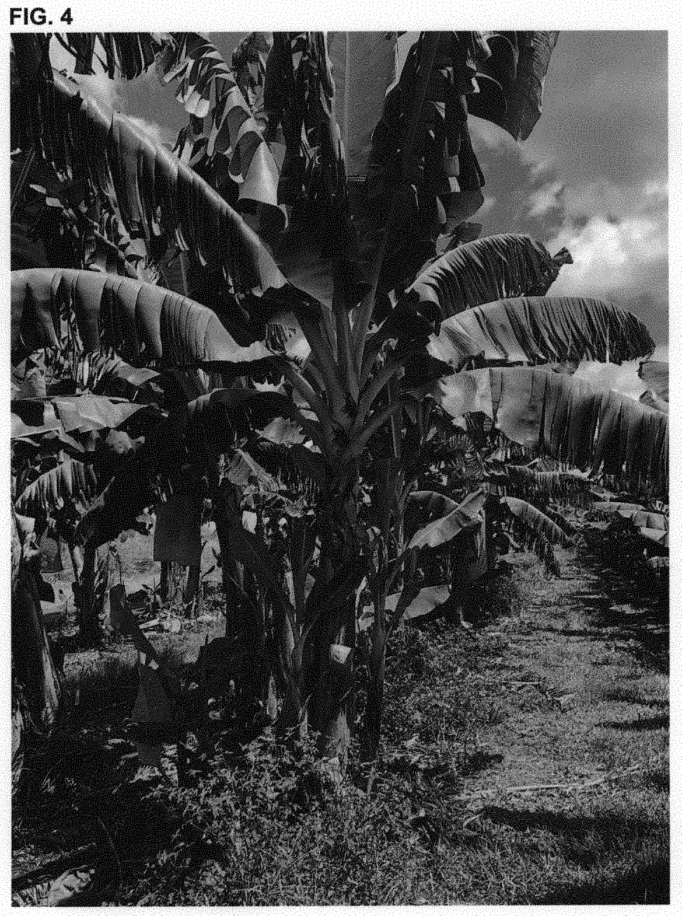

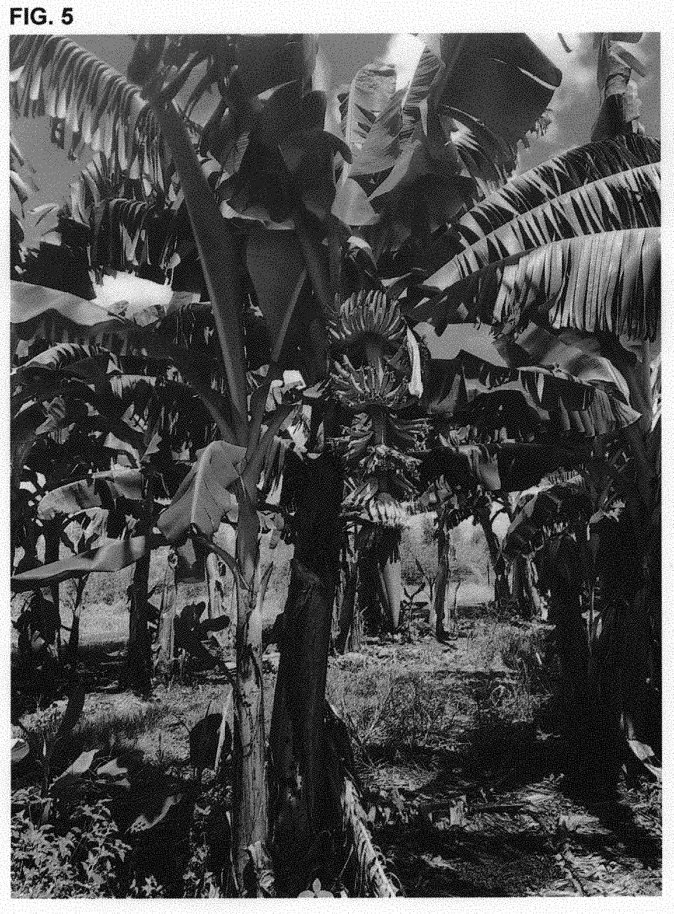

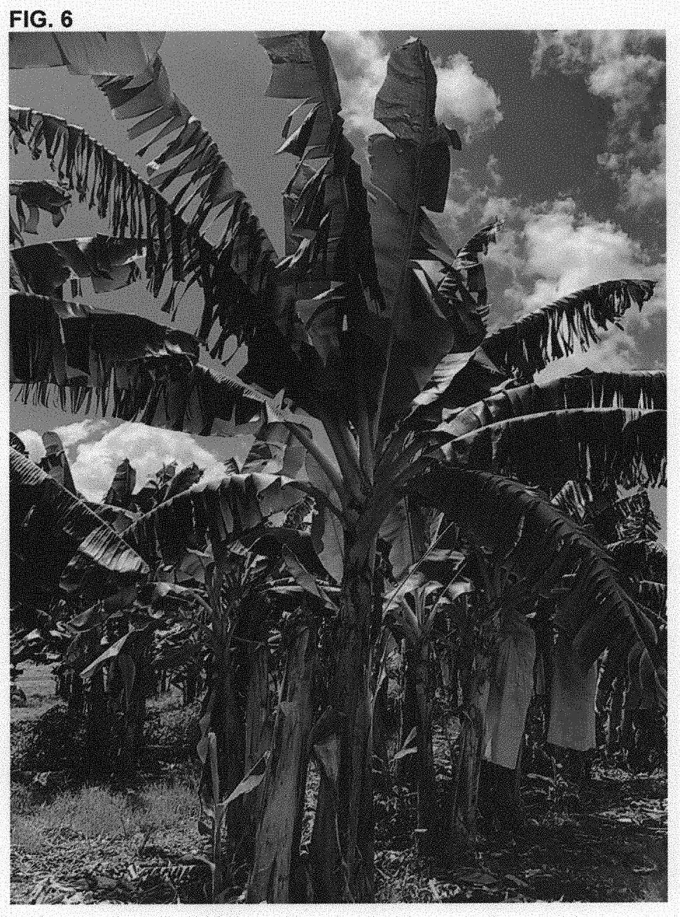

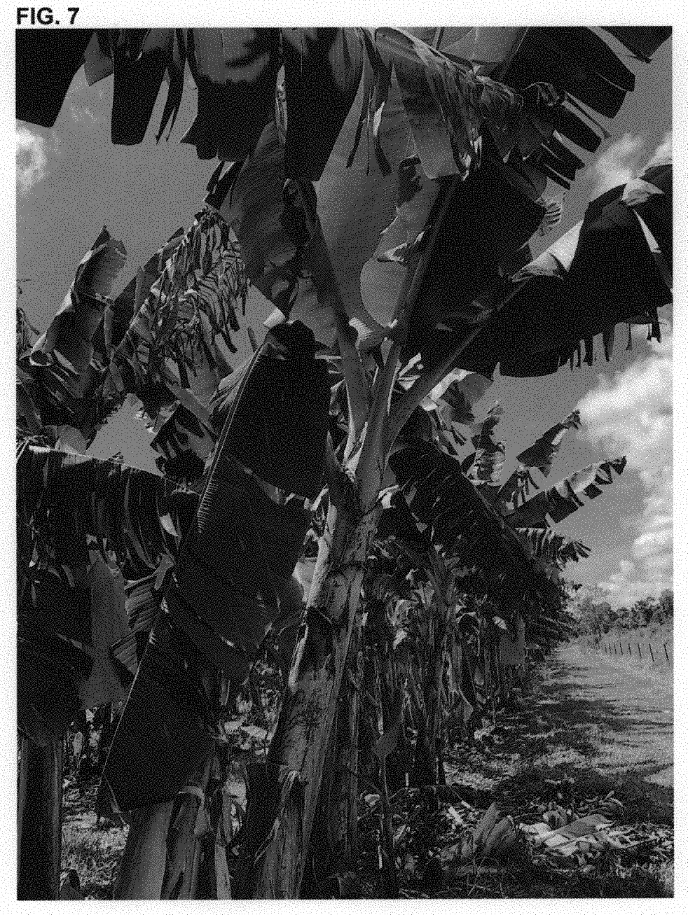

Genome sequencing of ‘QCAV-4’ was performed and the resulting sequence analyzed using Geneious software to characterize T-DNA insertion patterns and presence/absence of vector backbone. As illustrated in FIG. 2:
1. No vector backbone in ‘QCAV-4’.
2. Four complete copies of T-DNA insertion predicted to be present in a single intergenic locus on chromosome 6.
3. Random filler sequences of differing lengths between the four tandemly integrated T-DNAs (e, f and g) and the T-DNA>< banana genome junctions (c and d). These filler sequences also contain stretches of CaMV 35S promoter (driving the npt ORF in this T-DNA) and other sequences from the T-DNA including a small stretch (<50 bp) from RGA2 ORF
4. T-DNA-1><Chr6:299392** junction (k) was successfully amplified using multiple primers. Similarly T-DNA-4><Chr6:299394** junction (1) was also successfully amplified using multiple primers.
5. PCR amplified bands of three differing lengths (h, i and j) were obtained by using two pairs of primers binding across the three RB:LB junctions (e, f and g).

Sequencing of h, i and j PCR products revealed that the number of T-DNAs is neither 4 nor 5, rather something in between. At least one of these T-DNA is broken within the RGA2 CDS. In addition, not all the T-DNAs are arranged in direct repeats and there is at least one T-DNA: T-DNA junction which is inverted.

We claim:

1. A new and distinct variety of banana plant, substantially as herein shown and described.
